(19) United States
(12) Patent Application Publication
(10) Pub. No.: US 2015/0042643 A1
(43) Pub. Date: Feb. 12, 2015

(54) MEDICAL X-RAY APPARATUS

(71) Applicants: Koichi Shibata, Kyoto (JP); Yukio Mishina, Kyoto (JP); Kazuhiro Mori, Kyoto (JP)

(72) Inventors: Koichi Shibata, Kyoto (JP); Yukio Mishina, Kyoto (JP); Kazuhiro Mori, Kyoto (JP)

(73) Assignee: SHIMADZU CORPORATION, Kyoto (JP)

(21) Appl. No.: 14/388,137

(22) PCT Filed: Mar. 29, 2012
(86) PCT No.: PCT/JP2012/002198

§ 371 Date: Sep. 25, 2014

(51) Int. Cl. A61B 6/00; G06T 15/08; A61B 6/02; G06T 19/20

(52) U.S. Cl. CPC A61B 6/5235; G06T 19/20; G06T 15/08; A61B 6/022; G06T 2219/2008
USPC 345/419

(57) ABSTRACT

Provided is one example of a medical X-ray apparatus used as a C-arm fluoroscopy apparatus for endoscopy. Firstly, a three-dimensional image (CBCT volume data) is obtained through cone beam CT imaging (CBCT imaging). Then a stereogram (right and left fluoroscopy images) is generated through endoscopy (fluoroscopy). Thereafter, a stereoscopic image (right and left CBCT images) is generated based on the three-dimensional image (CBCT volume data) in projection directions in the stereogram. The three-dimensional image (stereoscopic image) and the stereogram are same in terms of an X-ray image. Accordingly, these images are superimposed to be displayed in real time on a display unit. This allows identification of a position and a direction of an object under fluoroscopy. Moreover, a three-dimensional coordinate of the object is detected from the stereogram in real time. Consequently, the position and the direction under fluoroscopy can be identified much readily, leading to perform accurate navigation.

### TECHNICAL FIELD

The present invention relates to a medical X-ray apparatus configured to display a fluoroscopy image in real time based on detected X-rays for diagnosis or treatment. More particularly, the present invention is directed to a technique of performing fluoroscopy for diagnosis or treatment while inserting an inserting member into a body of a subject to which the diagnosis or treatment is conducted.

### BACKGROUND ART

Examples of the inserting member include a bronchial endoscope used for endoscopy, a catheter or a wire used for orthopedic surgery or blood vessel contrast radiography, an applicator (a source-inserting applicator) configured to insert a source for a radiation treatment plan, and a dummy source. In the endoscopy, the bronchial endoscope or a clamp for biopsy inserted via the bronchial endoscope is inserted into a bronchus of the subject for bronchial diagnosis. In the blood vessel contrast radiography, the catheter or the wire is inserted into a blood vessel to a target site for diagnosis or treatment. In the radiation treatment plan, the source-inserting applicator and the dummy source are inserted to a treatment site for a treatment plan with the source. The following is description taking endoscopy as one example.

Prior to endoscopy, a three-dimensional bronchial image (a virtual endoscopic image) are preferably generated in accordance with three-dimensional data acquired through X-ray Computed Tomography (CT). Thereafter, in the course of inserting a bronchial endoscope into a bronchus of a subject to a given diagnosis position of the bronchus, an image (a bronchoscopic image) seen from a lumen of the bronchus is generated, and the image is displayed in real time, whereby endoscopy is performed to introduce (navigate) the tip of the bronchial endoscope. At this time, an important point is to determine an actual position of the tip of the bronchial endoscope from the virtual endoscopic image.

An image (an analogous image) analogous to the current bronchoscopic image is selected from the virtual endoscopic image. An actual position of the tip of the bronchial endoscope is then checked and determined with reference to the virtual endoscopic image for identification. See, for example, Patent Literature 1. Patent Literature 1 also discloses identification of the position with electromagnetism.

In the bronchial endoscopy for a peripheral lesion, a bronchial endoscope enters from a right main bronchial to a superior lobe of a right lung, and thereafter enters into a narrow peripheral bronchial. The bronchial endoscope has a diameter of 5 mm, whereas the narrow peripheral bronchus has a diameter of 1 mm, for example. Accordingly, the bronchial endoscope with a 5 mm diameter cannot be inserted into the narrow bronchus with a 1 mm diameter. In addition, when the thick bronchial endoscope is inserted into the thin bronchus, the endoscope merely travels to an insertable position. Consequently, only the insertable position of the lumen of the bronchus can be identified from the image (i.e., the bronchoscopic image) seen from the lumen of the bronchus. This causes impossible identification of the lumen of the thin bronchus.

Accordingly, a clamp is inserted into an opening of a treatment channel (clamp channel) provided at the tip of the bronchial endoscope. A position of the clamp is determined with the virtual endoscopic image obtained through X-ray CT. Thereafter, the clamp is introduced to the lesion (e.g., a tumor) to collect a sample such as a tissue. When a bronchial endoscope having an ultrafine diameter is used, the endoscope is insertable into a relatively thin bronchus. At this time, the endoscope is useful with a virtual endoscopic image that promotes understanding in direction of a bifurcation of the bronchus. However, it is not always possible for the endoscope to enter into the thin bronchus as a target. Moreover, the virtual endoscopic image may be helpful for a bronchus having a certainly thickness when the bronchial endoscope with a normal thickness is inserted.

### PATENT LITERATURE

Patent Literature 1 Japanese Patent Publication No. 2009-56239A

### SUMMARY OF INVENTION

### Technical Problem

However, a method of identifying a position of the tip by selecting the analogous image is difficult since a human tissue or structure is flexible. Such a problem may arise. Specifically, the bronchoscopic image differs from the analogous image obtained with X-rays in mode of display. The bronchoscopic image is displayed in real time. For instance, a bronchial endoscope image is displayed every time a tissue or a structure moves while a human breathes. On the other hand, the analogous image is not displayed in real time. Accordingly, the analogous image is displayed only with a figure in a certain phase. Consequently, it is difficult to match the both images to each other, leading to difficulty in identifying the position of the tip by selecting the analogous image. Moreover, in the endoscope image, mucus is transparent through which a mucous membrane is visible. On the other hand, in the analogous image with X-rays, it is difficult to differentiate between mucous and a mucous membrane.

The method of identifying the position electromagnetically allows determining an absolute position of the tip. On the other hand, the method has difficulty in determining a relationship between the tip and a peripheral anatomical structure as well as a direction (i.e., an inserting direction) of the tip. Such a problem may also arise. The above problems cause difficulty of accurate guidance (i.e., navigation).

The present invention has been made regarding the state of the art noted above, and its object is to provide a medical X-ray apparatus that allows accurate navigation.

### Solution to Problem

To fulfill the above object, Inventors have made intensive research and attained the following findings.

Specifically, attention has been focused on a three-dimensional image (a virtual endoscopic image) for an image displayed in real time independently of a bronchoscopic image with a currently-used endoscope, the three-dimensional image being obtained in advance based on X-rays. In this case, when a fluoroscopy image based on X-rays is adopted as the image displayed in real time, the three-dimensional image is same as the fluoroscopy image in terms of an X-ray image. Consequently, an object position (a tip of the endoscope in the endoscopy) can be identified, and thus accurate navigation is obtainable by the fluoroscopy while the inserting member, typified by a bronchial endoscope, is inserted into the subject. Such finding has been obtained.

Moreover, when a stereogram formed by two fluoroscopy images with parallax in projection directions is adopted, a three-dimensional coordinate can be identified based on the stereogram (displayed in real time), and an inserting direction is also determined. Such finding has also been obtained. Furthermore, another finding has been obtained that the three-dimensional coordinate can be identified and the inserting direction can be determined based on the fluoroscopy images (displayed in real time) and the three-dimensional image without adopting the stereogram.

The present invention based on the above findings adopts the following configuration. Specifically, one embodiment (the former embodiment) of the present invention discloses a medical X-ray apparatus configured to perform diagnosis or treatment by displaying a fluoroscopy image in real time in accordance with detected X-rays. The medical X-ray apparatus includes a stereogram generating device configured to generate a stereogram formed by two fluoroscopy images with parallax in projection directions; a stereoscopic image generating device configured to generate a stereoscopic image in the projection directions in the stereogram generated by the stereogram generating device based on a three-dimensional image obtained in advance in accordance with X-rays; a superimposing device configured to perform superimposition of the stereogram in the projection directions on the stereoscopic image generated by the stereoscopic image generating device; a display device configured to display an image subjected to the superimposition in real time by the superimposing device; and a three-dimensional coordinate detecting device configured to calculate and detect a three-dimensional coordinate of an object from a position of the object displayed on a screen of the display device in real time in accordance with the stereogram generated by the stereogram generating device.

The stereogram generating device of the medical X-ray apparatus according to the embodiment (the former embodiment) of the present invention generates the stereogram, the stereogram being formed by the two fluoroscopy images (obtained based on X-rays) with parallax in the projection directions. The stereoscopic image generating device generates the stereoscopic image based on the three-dimensional image in the projection directions of the stereogram generated by the stereogram generating device, the three-dimensional image being obtained in advance based on X-rays.

The superimposing device performs superimposition of the stereogram in the projection directions on the stereoscopic image generated by the stereoscopic image generating device. The image subjected to the superimposition by the superimposing device is displayed on the display device in real time. The three-dimensional coordinate detecting device calculates and detects the three-dimensional coordinate of the object from a position of the object displayed on the screen of the display device in real time in accordance with the stereogram generated by the stereogram generating device. As noted above, the three-dimensional image (the stereoscopic image) and the fluoroscopy image (the stereogram) are same in terms of an X-ray image. Accordingly, superimposing these images to display a superimposed image in real time allows identification of the position and the direction of the object under fluoroscopy. Moreover, the three-dimensional coordinate is detected from the stereogram in real time, facilitating identification of the position and the direction of the object under fluoroscopy. Consequently, accurate navigation is obtainable.

Moreover, another embodiment (the latter embodiment) of the present invention discloses a medical X-ray apparatus configured to perform diagnosis or treatment by displaying a fluoroscopy image in real time in accordance with on detected X-rays. The medical X-ray apparatus includes a region of interest setting device configured to set a local region of interest; an image shifting device configured to (1) shift a stereoscopic image in a projection direction of the fluoroscopy image in the region of interest set by the region of interest setting device in synchronization with shifting of the fluoroscopy image, the stereoscopic image being based on a three-dimensional image obtained in advance in accordance with the X-rays, or configured to (2) shift the fluoroscopy image in the region of interest set by the region of interest setting device in synchronization with a position of a stereoscopic image in a projection direction of the fluoroscopy image, the stereoscopic image being fixed and being based on a three-dimensional image obtained in advance in accordance with the X-rays; a superimposing device configured to perform superimposition of (1) the fluoroscopy image on the stereoscopic image shifted by the image shifting device, or (2) the stereoscopic image on the fluoroscopy image shifted by the image shifting device in the region of interest; a display device configured to display an image subjected to the superimposition by the superimposing device in real time; and a three-dimensional coordinate detecting device configured to calculate and detect a three-dimensional coordinate of an object from a position of the object displayed on a screen of the display device in real time in accordance with the three-dimensional image and the fluoroscopy image in the region of interest.

In one embodiment (the latter embodiment) of the present invention, the region of interest setting device of the medical X-ray apparatus sets a local region of interest. The image shifting device (1) shifts the stereoscopic image in the projection direction of the fluoroscopy image (obtained in accordance with the X-rays) in the region of interest set by the region of interest setting device in synchronization with shifting of the fluoroscopy image. The stereoscopic image is based on the three-dimensional image obtained in advance in accordance with the X-rays. Alternatively, the image shifting device (2) shifts the fluoroscopy image in the region of interest set by the region of interest setting device in synchronization with the position of the stereoscopic image fixed and based on the three-dimensional image obtained in advance in accordance with the X-rays in the projection direction of the fluoroscopy image. Typically, a tissue or a structure inside the body contracts and expands due to a body motion (e.g., a body motion by respiration) of the subject. In contrast to this, the contraction and expansion is not regarded within the local region of interest, and thus the image is shifted while the tissue or the structure has a constant size. Moreover, when fluoroscopy is conducted while the inserting member is inserted, the entire image is not so important, but only the region of interest is needed. Accordingly, in the above case (1), the stereoscopic image can be shifted in the region of interest in synchronization with shifting of the fluoroscopy image. Moreover, in the above case (2), the fluoroscopy image is shifted in synchronization with the position of the stereoscopic image fixed in the region of interest. Consequently, even when the fluoroscopy image is shifted, the fluoroscopy image is always located on the position of the stereoscopic image as if the fluoroscopic image is stable. For the body motion due to respiration, a three-dimensional image in synchronization with a respiration sensor or a three-dimensional image in synchronization with each of a plurality of respiratory phases is obtained in advance to deal with superimposition by the body motion. Such a mode is conceivable. However, the mode needs the respiration sensor, or increases radiographic frequency for obtaining an image for each of the phases, causing increase in inspection time, exposure radiation dose, or processing time. Consequently, the mode is not practical. In addition, large movement of the subject causes retaking all the images, leading to much waste. The latter embodiment differs from a mode in which a deviation amount of the stereoscopic image is calculated in accordance with a deviation amount of the fluoroscopy image upon changing the projection direction to superimpose both the images. In the latter embodiment, the stereoscopic image (or the fluoroscopy image in the above case (2)) is simply shifted under an assumption that the tissue or the structure has a constant size in the region of interest. Accordingly, the currently-used respiration sensor is not needed. In addition, a radiographic frequency, an exposure radiation dose, an inspection time, and a processing time can be reduced without obtaining the three-dimensional image in advance in synchronization with each of a plurality of respiratory phases.

The superimposing device then superimposes (1) the fluoroscopy image on the stereoscopic image shifted by the image shifting device in the region of interest. Alternatively, the superimposing device superimposes (2) the stereoscopic image on the fluoroscopy image shifted by the image shifting device in the region of interest. Moreover, an image obtained by superimposing by the superimposing device is displayed in real time on the display device. The three-dimensional coordinate detecting device calculates and detects a three-dimensional coordinate of the object from the position on the screen of the object displayed in real time in accordance with the three-dimensional image and the fluoroscopy image in the region of interest. As noted above, both the three-dimensional image (stereoscopic image) and the fluoroscopy image are same in terms of an X-ray image. Accordingly, these images are superimposed on each other to be displayed in real time, whereby a position and a direction of the object under fluoroscopy can be identified. Moreover, the three-dimensional coordinate is detected from the three-dimensional image and the fluoroscopy image in real time. This facilitates identification of the position and the direction of the object under fluoroscopy, causing accurate navigation.

The latter embodiment of the present invention preferably includes a region of interest re-setting device configured to reset a region of interest so as to contain the three-dimensional coordinate displayed in real time when the three-dimensional coordinate goes beyond the region of interest. The image shifting device, the superimposing device, the display device, and the three-dimensional coordinate detecting device each repeatedly perform its processing to the region of interest reset by the region of interest resetting device. This achieves navigation while tracking the variable three-dimensional coordinate for example when fluoroscopy is conducted with the inserting member being inserted. Moreover, the region of interest also tracks the coordinate during the navigation while being reset repeatedly. This achieves accurate navigation while tracking the coordinate.

Moreover, combination of the former and the latter embodiments is allowable.
Specifically, the latter embodiment of the present invention includes a stereogram generating device configured to generate a stereogram formed by two fluoroscopy images having parallax in projection directions; and a stereoscopic image generating device configured to generate a stereoscopic image based on a three-dimensional image in the projection directions of the stereogram generated by the stereogram generating device. The image shifting device (1) shifts the stereoscopic image generated by the stereoscopic image generating device in the region of interest in synchronization with shifting of the stereogram, or (2) shifts the stereogram in synchronization with a position of the stereoscopic image in the region of interest, the stereoscopic image being fixed by the stereoscopic image generating device. The superimposing device performs superimposition of (1) the stereogram on the stereoscopic image shifted by the image shifting device for every projection direction, or (2) the stereoscopic image on the stereogram shifted by the image shifting device for every projection direction in the region of interest. The display device displays an image subjected to the superimposition by the superimposing device in real time. The three-dimensional coordinate detecting device calculates and detects the three-dimensional coordinate based on the three-dimensional image and the stereogram in the region of interest.

According to the combination of the former and latter embodiments of the present invention, the latter includes the stereogram generating device and the stereoscopic image generating device that are same as those in the former. The fluoroscopy image is restricted to the stereogram in the image shifting device of the latter. Accordingly, the image shifting device shifts the stereoscopic image in the region of interest generated by the stereoscopic image generating device in accordance with shifting of the stereogram in the case (1), or fixes the stereoscopic image, and then shifts the stereogram in accordance with positions of the fixed stereoscopic image in the case (2). The fluoroscopy image is restricted to the stereogram in the superimposing device of the latter. Accordingly, the superimposing device superimposes the stereogram (the stereoscopic image in the case (2)) on the stereoscopic image (the stereogram in the case (2)) shifted by the image shifting device in the region of interest for every projection direction. In other words, the superimposing device of the former restricts a processed portion to the region of interest. Consequently, the stereogram (the stereoscopic image in the case (2)) is superimposed on the stereoscopic image (the stereogram in the case (2)) shifted by the image shifting device in the region of interest for every projection direction.

Similar to the display device in the former embodiment, the display device in the latter embodiment displays the image in real time that is subjected to the superimposition by the superimposing device. In addition, the three-dimensional coordinate detecting device in the latter embodiment restricts the fluoroscopy image to the stereogram. Consequently, the three-dimensional coordinate detecting device calculates and detects the three-dimensional coordinate based on the three-dimensional image and the stereogram in the region of interest. In other words, the three-dimensional coordinate detecting device in the former embodiment restricts a processed portion to the region of interest, and adds the three-dimensional image, besides the stereogram, to basis data. Consequently, the three-dimensional coordinate is calculated and detected based on the three-dimensional image and the stereogram in the region of interest. Since the other operations and effects are produced from the combination of the former and latter embodiments, a description thereof is to be omitted.

One example of the stereogram generating device generates the stereogram formed by two fluoroscopy images. The fluoroscopic images are obtained through fluoroscopy in real rime with parallax in the projection directions. That is, the fluoroscopy for stereogram obtains the two fluoroscopy images with parallax in real time for every fluoroscopy, whereby the stereogram is generated.

Another example of the stereogram generating device generates the stereogram from one original fluoroscopy image obtained through fluoroscopy in real time. The stereogram is formed by the original fluoroscopy image and a fluoroscopy image based on the three-dimensional image with parallax in a projection direction of the original fluoroscopy image. That is, typical fluoroscopy (not fluoroscopy for stereogram) obtains one original fluoroscopy image in real time for every fluoroscopy. Then the stereogram is generated from the original fluoroscopy image, the stereogram being formed by the original fluoroscopy image and the fluoroscopy image with parallax in the projection direction of the original fluoroscopy image.

In the medical X-ray apparatus according to the embodiments, including the former and latter, of the present invention, the three-dimensional coordinate detecting device detects a position of a tip of an inserting member as the three-dimensional coordinate, the inserting member being inserted into a subject to which diagnosis or treatment is performed. When fluoroscopy is conducted while the inserting member, typified by a bronchial endoscope, a catheter wire, and a source-inserting applicator, is inserted, the position and direction of the inserting member can be identified readily under the fluoroscopy without electricity and magnetism currently used. Here, examples of the inserting member include an endoscope, a source-inserting applicator, a dummy source, and a catheter wire.

### Advantageous Effects of Invention

With the medical X-ray apparatus (the former embodiment) of the present invention, both the three-dimensional image (stereoscopic image) and the fluoroscopy image are same in terms of an X-ray image. Accordingly, these images are superimposed on each other to be displayed in real time, whereby a position and a direction of the object under the fluoroscopy can be identified. Moreover, the three-dimensional coordinate is detected from the stereogram in real time. This facilitates identification of the position and direction of the object under the fluoroscopy, causing accurate navigation.

With the medical X-ray apparatus (the latter embodiment) of the present invention, both the three-dimensional image (stereoscopic image) and the fluoroscopy image are same in terms of an X-ray image. Accordingly, these images are superimposed on each other to be displayed in real time, whereby a position and a direction of the object under the fluoroscopy can be identified. Moreover, the three-dimensional coordinate is detected from the three-dimensional image and the fluoroscopy image in real time. This facilitates identification of the position and direction of the object under the fluoroscopy, causing accurate navigation.

### BRIEF DESCRIPTION OF DRAWINGS

FIG. 1 is a schematic view and a block diagram of a C-arm fluoroscopy apparatus according to one embodiment.

FIG. 2(a) is a schematic view of cone-beam CT imaging (CBCT imaging) with the C-arm fluoroscopy apparatus prior to endoscopy (fluoroscopy). FIG. 2(b) is a schematic view of the endoscopy (fluoroscopy) with the C-arm fluoroscopy apparatus.

FIG. 3 is a schematic view of a data flow for each image.

FIG. 4 is a schematic view for explanation of generating a stereoscopic image (right and left CBCT images) from CBCT volume data.

FIG. 5 is a schematic view of one aspect of an image display mode with a display unit.

FIG. 6 is a schematic view of a bronchial endoscope.

FIG. 7 is a flow chart illustrating a series of navigations according to another embodiment.

FIG. 8(a) to (c), FIG. 9(a) to (c), FIG. 10(a) to (c), and FIG. 11 are schematic views each illustrating one aspect of a display unit according to the other embodiment.

FIG. 12 is a schematic view for explanation of generating a stereoscopic image (right and left CBCT images) from CBCT volume data according to one modification of the present invention.

FIG. 13 is a schematic view of a C-arm fluoroscopy apparatus adopting a typical X-ray tube 2 with one focus according to the modification. FIG. 13(a) is a schematic view of cone-beam CT imaging (CBCT imaging) with the C-arm fluoroscopy apparatus prior to endoscopy (fluoroscopy), and FIG. 13(b) is a schematic view of the endoscopy (fluoroscopy) with the C-arm fluoroscopy apparatus.

### EMBODIMENT 1

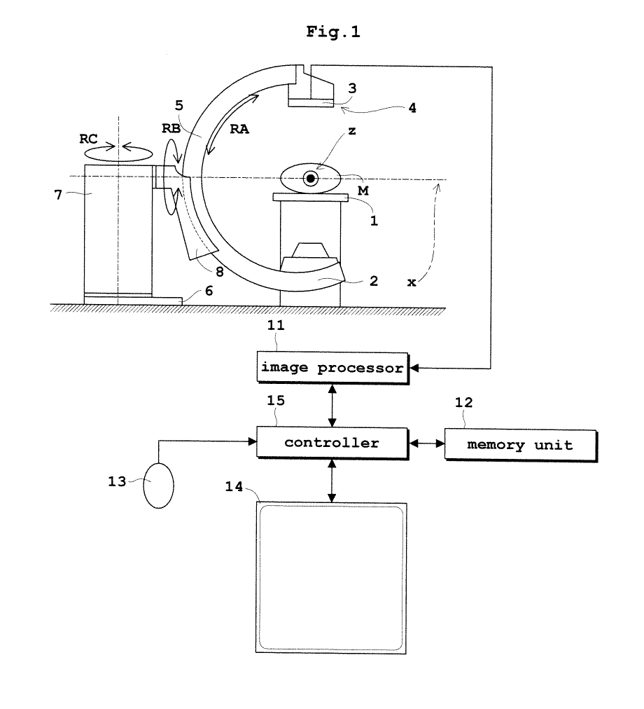

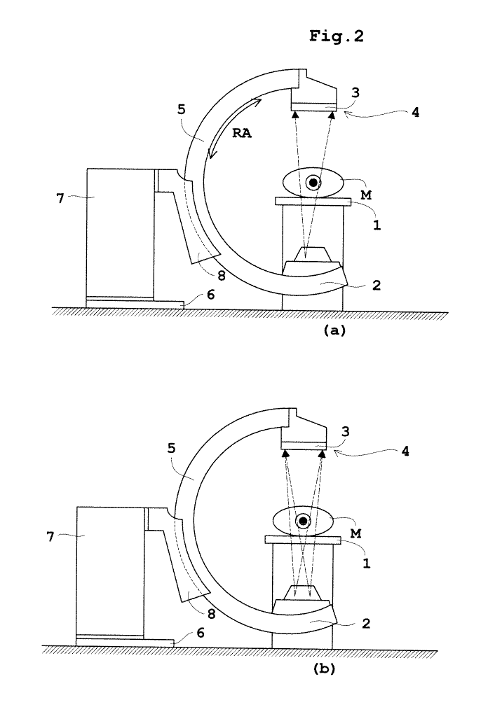

The following describes Embodiment 1 with reference to drawings. FIG. 1 is a schematic view and a block diagram of a C-arm fluoroscopy apparatus according to each embodiment. FIG. 2(a) is a schematic view of cone-beam CT imaging (CBCT imaging) with the C-arm fluoroscopy apparatus prior to endoscopy (fluoroscopy). FIG. 2(b) is a schematic view of the endoscopy (fluoroscopy) with the C-arm fluoroscopy apparatus. Embodiment 1 as well as Embodiments 2 and 3 describe the C-arm fluoroscopy apparatus as one example of a medical X-ray apparatus and the endoscope as one example of an inserting member.

As illustrated in FIG. 1, a C-arm fluoroscopy apparatus according to Embodiment 1 as well as Embodiments 2 and 3 moves independently of a top board 1 for supporting a subject M placed thereon. The C-arm fluoroscopy apparatus includes an imaging system 4 composed of an X-ray tube 2 and an X-ray detector 3. In Embodiment 1 as well as Embodiments 2 and 3, the X-ray tube 2 is one vessel (a stereo X-ray tube) having two focuses. Specifically, as illustrated in FIG. 2, the focuses are switchable by pulses. Right and left fluoroscopy images are displayed in real time through switchingly emitting X-rays alternately from side to side.

The C-arm fluoroscopy apparatus further includes a C-arm 5 having a first end for holding the X-ray tube 2 and a second end for holding the X-ray detector 3. The C-arm 5 is curved in a direction of a rotational central axis x. The C-arm 5 rotates along itself around a body axis z of the subject M (i.e., in a direction denoted by an arrow RA). Accordingly, the X-ray tube 2 and the X-ray detector 3 held with the C-arm 5 are rotatable in the same direction. The C-arm 5 also rotates around the rotational central axis x (i.e., in a direction denoted by an arrow RB) that is orthogonal to the body axis z. Accordingly, the X-ray tube 2 and the X-ray detector 3 held with the C-arm 5 are rotatable in the same direction.

Specifically, the C-arms 5 is held via a strut 7 and arm holder 8 on a base 6 that is fixedly arranged on the floor. The strut 7 is rotatable relative to the base 6 around a vertical axis (i.e., in a direction denoted by an arrow RC). The imaging system 4 is rotatable in the same direction along with the C-arm 5 held with the strut 7. Moreover, the arm holder 8 is held so as to rotate relative to the strut 7 in the rotational central axis x. Accordingly, the imaging system 4 is rotatable along with the C-arm 5 held on the arm holder 8 in the same direction. Furthermore, the C-arm 5 is held so as to rotate relative to the arm holder 8 around the body axis z of the subject M. Accordingly, the imaging system 4 is rotatable in the same direction along with the C-arm.

As illustrated in FIG. 1, the C-arm fluoroscopy apparatus further includes an image processor 11, a memory unit 12, an input unit 13, a display unit 14, and a controller 15. The image processor 11 performs various types of image processing in accordance with X-rays detected with the X-ray detector 3. The memory unit 12 writes and stores data on various images (e.g., CBCT volume data, a stereoscopic image, and an image subjected to superimposition in each embodiment) obtained with the image processor 11. The input unit 13 inputs data or instructions. The display unit 14 displays a fluoroscopy image, a CBCT image, or an image obtained by superimposing these images. The controller 15 controls these units en block. In addition to these units, the C-arm fluoroscopy apparatus includes a high voltage generating unit configured to generate high voltages to apply a tube current or a tube voltage to the X-ray tube 2. Since the unit does not correspond to the feature of the present invention nor has no construction associated with the feature of present invention, the unit is not shown. Here, the image processor 11 corresponds to the stereogram generating device, the stereoscopic image generating device, and the superimposing device in the present invention. The display unit 14 corresponds to the display device in the present invention. The controller 15 corresponds to the three-dimensional coordinate detecting device in the present invention.

The image processor 11 sends a projected image as a fluoroscopy image via the controller 15 to the display unit 14 upon endoscopy (fluoroscopy) where the fluoroscopy image is displayed in real time. Here, the projected image is generated in accordance with X-rays detected with the X-ray detector 3. The display unit 14 displays the fluoroscopy image in real time, whereby an operator achieves monitors the fluoroscopy image in real time.

In Embodiment 1 as well as Embodiment 3 to be mentioned later, as illustrated in FIG. 2(b), a focus is switched through pulses from the X-ray tube 2. Accordingly, X-rays are alternately applied from side to side. The X-ray detector 3 detects the X-rays and two projected images are generated in accordance with the X-rays. The image processor 11 adopts the projected images as two fluoroscopy image (right and left fluoroscopy images) with parallax in projection directions. That is, the image processor 11 generates a stereogram formed by two fluoroscopy image (the right and left fluoroscopy images) obtained through fluoroscopy in real time with parallax in the projection directions.

In Embodiment 1 as well as Embodiments 2 and 3 mentioned later, Cone-Beam CT imaging (CBCT imaging) is performed by moving the imaging system 4 in each direction (e.g., by rotating by 200 degrees in the direction denoted by the arrow RA in FIGS. 1 and 2(a)), and emitting cone-beam (CB) X-rays from one focus to detect the X-rays with the X-ray detector 3 prior to the endoscopy (fluoroscopy), as illustrated in FIG. 2(a).

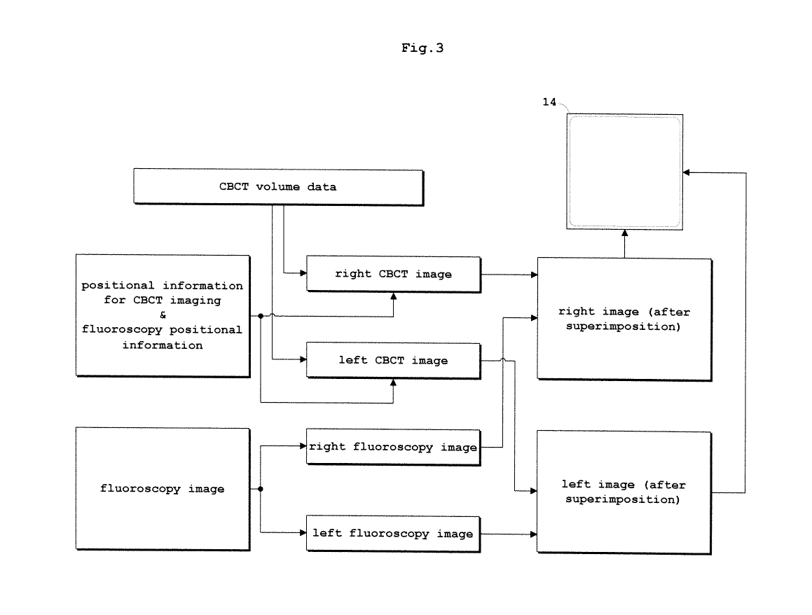

A plurality of projected images is collected by moving the imaging system 4 in every direction. Upon the CBCT imaging prior to the endoscopy (fluoroscopy), the image processor 11 performs three-dimensional reconstruction to the plurality of projected images to generate a three-dimensional image (CBCT volume data). Moreover, the image processor 11 generates right and left CBCT images (see FIGS. 3 to 5), which are each to be described as a stereoscopic image, in accordance with the three-dimensional image (the CBCT volume data). The memory unit 12 writes and stores the CBCT volume data and the stereoscopic image (the right and left CBCT images) via the controller 15. A concrete three-dimensional reconstruction method (calculation method) and a concrete generating method (calculation method) of generating the stereoscopic image (the right and left CBCT images) do not correspond to the feature of the present invention, and thus a description thereof is to be omitted.

Moreover, the image processor 11 superimposes the stereogram (right and left fluoroscopy images) in every projection direction on the stereoscopic image (the right and left CBCT images). Specifically, the right fluoroscopy image is superimposed on the right CBCT image, and the left fluoroscopy image is superimposed on the left CBCT image. Accordingly, images subjected to the superimposition (images after the superimposition) are generated. The memory unit 12 writes and stores the images via the controller 15.

The memory unit 12 writes and stores data such as the CBCT volume data generated by the image processor 11, the stereoscopic image (the right and left CBCT images), and the images after superimposition via the controller 15. The memory unit 12 reads the data where appropriate, and transmits the data via the controller 15 to the display unit 14 where the data is displayed. The memory unit 12 is composed of a storage medium typified by a ROM (Read-only Memory), a RAM (Random-Access Memory), and a hard disk. In Embodiment 1 as well as Embodiments 2 and 3 mentioned later, the stereoscopic image (the right and left CBCT images) and the images after the superimposition are read out from the memory unit 12 upon the endoscopy (fluoroscopy) to be displayed on the display unit 14.

The input unit 13 transmits the data or the instructions inputted by the operator to the controller 15. The input unit 13 is formed of a pointing device typified by a mouse, a keyboard, a joystick, a trackball, and a touch panel.

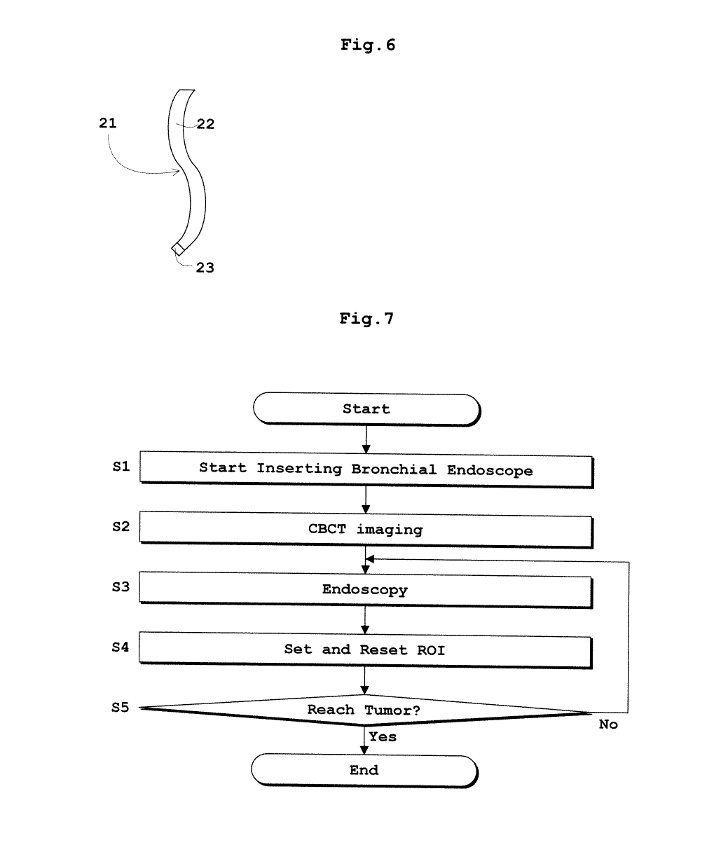

The display unit 14 is constituted by a monitor. In Embodiment 1 as well as Embodiments 2 and 3 mentioned later, the display unit 14 is constituted by a 3D display such as a 3D monitor configured to display a pair of images three-dimensionally or a binocular head mounted display (a two-screen head-mounted display). Concrete display is to be mentioned later in FIG. 5.

The controller 15 controls the above units en block that constitute an X-ray blood vessel photographing apparatus. Embodiment 1 as well as Embodiments 2 and 3 mentioned later each have a function of calculating and detecting a three-dimensional coordinate of an object from a position of the object (in each embodiment, a position of a tip of the bronchial endoscope) displayed on a screen of the displaying unit 14 in real time. Especially in Embodiment 1, the controller 15 calculates and detects the three-dimensional coordinate in accordance with the stereogram (the right and left fluoroscopy images) generated by the image processor 11. The image processor 11 and the controller 15 are constituted by a central processing unit (CPU). Via the controller 15, the data on the images each obtained by the image processor 11 is written and stored in the memory unit 12 or is transmitted to the display unit 14 to be displayed.

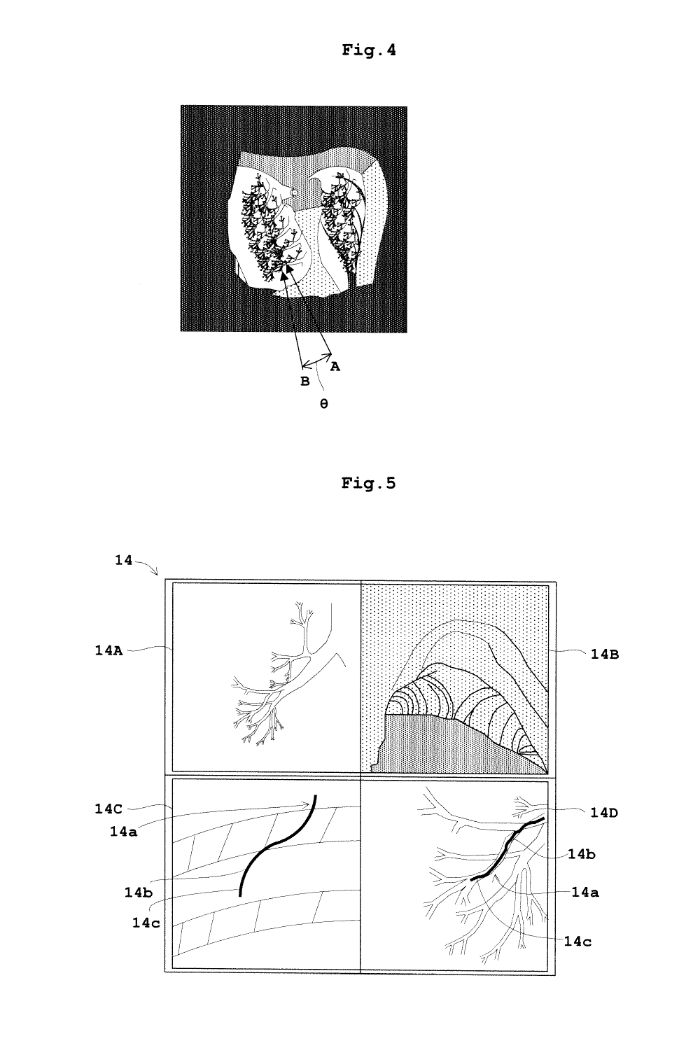

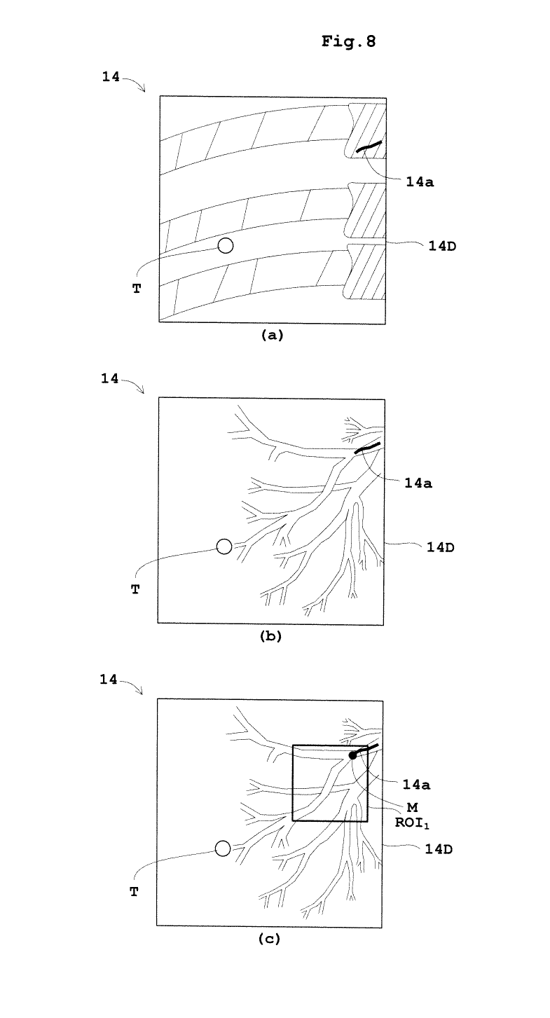

The following describes generation and display of each image with reference to FIGS. 3 to 6. FIG. 3 is a schematic view of a data flow for each image. FIG. 4 is a schematic view for explanation of generating the stereoscopic image (right and left CBCT images) from the CBCT volume data. FIG. 5 is a schematic view of one aspect of displaying the image with a display unit. FIG. 6 is a schematic view of the bronchial endoscope.

In FIG. 4, a projection direction in which the right CBCT image is generated is denoted by an “A” direction, and a projection direction in which the left CBCT image is generated is denoted by a “B” direction. Moreover, a projection direction of the right fluoroscopy image displayed in real time is also denoted by the A direction, and a direction with parallax relative to the A direction is also denoted by the B direction. Under such a condition, a fluoroscopy image obtained in the B direction corresponds to a left fluoroscopy image. That is, a relative angle θ between the projection directions (the A and B directions) in the right and left CBCT images, respectively, depends on a fluoroscopy angle of the C-arm 5 (see FIGS. 1 and 2). In a crossing method, the relative angle θ is approximately from 5 to 10 degrees. Consequently, as illustrated in FIG. 3, the right and left CBCT images can be generated from the three-dimensional image (the CBCT volume data) in accordance with positional information for CBCT imaging and fluoroscopy positional information from the X-ray tube 2 as the stereo X-ray vessel (see FIGS. 1 and 2).

More specifically, as illustrated in FIG. 3, the image processor 11 (see FIG. 1) generates the three-dimensional image (the CBCT volume data) based on a plurality of projection images obtained through the cone-beam CT imaging (the CBCT imaging) prior to the endoscopy (fluoroscopy). The generated CBCT volume data is written and stored via the controller 15 (see FIG. 1) to the memory unit 12 (see FIG. 1).

Thereafter, the image processor 11 reads out the CBCT volume data obtained in advance (and stored in the memory unit 12) (via the controller 15) upon the endoscopy (fluoroscopy). Then the image processor 11 generates the stereoscopic image (the right and left CBCT images) from the CBCT volume data in every projection direction (the A and B directions in FIG. 4) in the stereogram (the right and left fluoroscopy images) generated by the same image processor 11. That is, the right and left CBCT images are each generated in accordance with the positional information by the CBCT imaging or the fluoroscopic positional information. The generated right and left CBCT images are written and stored via the controller 15 to the memory unit 12, or transmitted to the display unit 14 (see FIGS. 1, 3, and 5) to be displayed.

Moreover, the image processor 11 generates right and left fluoroscopy images upon the endoscopy (fluoroscopy), and superimposes the images with the right and left CBCT images, respectively, thereby generating images (right and left images) after the superimposition. For real-time display, the images are not written in the memory unit 12, but are transmitted to the display unit 14 via the controller 15 to be displayed upon the endoscopy (fluoroscopy). Direct display on the display unit 14 in this manner leads to real-time display of the images (the right and left images) after the superimposition on the display unit 14. On the other hand, the images may be written and stored in the memory unit 12 via the controller 15 for adopting the image (the right and left images) after the superimposition later.

As illustrates in FIG. 5, the display unit 14 includes four monitors. In FIG. 5, the display unit 14 is formed by a monitor 14A configured to display right and left right CBCT images (also referred to as an “operation planning image”), a monitor 14B configured to display an image (bronchoscopic image) viewed from a bronchial lumen, a monitor 14C configured to display right and left fluoroscopy images in real time, and a monitor 14D configured to display images (right and left images) after superimposition in real time.

In a 3D monitor that displays a pair of images three-dimensionally, the monitor 14C uses the right fluoroscopy image as one of images for right and left eyes (here, as an image for a right eye) on the 3D monitor, and the left fluoroscopy image as the other image for right and left eyes (here, as an image for a left eye). Moreover, the monitor 14D uses the image (right image), generated by superimposing the right fluoroscopy image on the right CBCT image, as one of images for right and left eyes (here, as an image for a right eye) on the 3D monitor, and the image (left image), generated by superimposing the left fluoroscopy image on the left CBCT image, as one of the images for right and left eyes (here, as an image for a left eye).

In a binocular head mounted display (two-screen head mounted display), the monitor 14C displays the right and left fluoroscopy image in parallel to use the images as a stereogram. The monitor 14D displays the image (right image), generated by superimposing the right fluoroscopy image on the right CBCT image, and the image (left image), generated by superimposing the left fluoroscopy image on the left CBCT image, in parallel to use the images as a stereogram. In the binocular head mounted display, a pair of images may be displayed on the right and left of the screen for an operator to perform stereoscopy. With such a construction, no special device, such as a 3D monitor, is required, and a currently-used apparatus configuration (a typical monitor) is available.

A bronchial endoscope 21 as illustrated in FIG. 6 is used for the endoscopy (fluoroscopy). The bronchial endoscope 21 includes a wire guide 22, and a tip 23 formed by a treatment channel through which a clamp for biopsy and an imaging element are inserted. Moreover, the display unit 14 may display an image in real time obtained with the imaging element of the bronchial endoscope 21 upon the endoscopy (fluoroscopy). The tip 23 is inserted through the guide 22 inside a body (an oral cavity and a bronchus) of the subject M (see FIGS. 1 and 2), whereby the bronchial endoscope 21 is inserted inside the body. Here, the bronchial endoscope 21 corresponds to the inserting member in the present invention.

The monitors 14C and 14D in FIG. 5 display the bronchial endoscope 21 in FIG. 6 in real time as a figure. In FIG. 5, an entire figure of the bronchial endoscope 21 is denoted by a numeral 14a, a figure of the guide 22 is denoted by a numeral 14b, and a figure of the tip 23 is denoted by a numeral 14c. The controller 15 (see FIG. 1) calculates and detects a three-dimensional coordinate of an object (the tip 23 of the bronchial endoscope 21) from a position of the object displayed in real time on the monitor in accordance with the stereogram (right and left fluoroscopy images). The FIG. 14c of the tip 23 as well as the FIG. 14b of the guide 22 has pixel values extremely different from those therearound. Consequently, the controller 15 can calculate and detect the three-dimensional coordinate automatically. Of course, the following may be adopted. That is, an operator recognizes the FIG. 14c of the tip 23, and manually inputs a position of the tip 23 through the input unit 13 (see FIG. 1) by putting a pointer on a position of the monitor corresponding to the FIG. 14c. The controller 15 then detects the three-dimensional coordinate base on the position. Moreover, combination of manual control and automatic control may be adopted.

In the C-arm fluoroscopy apparatus according to Embodiment 1, the stereogram generating device (the image processor 11 in Embodiment 1) generates the stereogram formed by the two fluoroscopy images (obtained based on X-rays) with parallax in the projection directions. The stereoscopic image generating device (the image processor 11 in Embodiment 1) generates the stereoscopic image (the right and left CBCT images in Embodiment 1) through the cone-beam CT imaging (CBCT imaging) prior to the endoscopy (fluoroscopy) based on the three-dimensional images (CBCT volume data) in the projection directions in the stereogram generated by the stereogram generating device (the image processor 11). Here, the three-dimensional image (the CBCT volume data in Embodiment 1) is obtained in advance based on the X-rays.

The superimposing device (the image processor 11 Embodiment 1) superimposes the stereogram in the projection directions (the right and left fluoroscopy images in Embodiment 1) on the stereoscopic image (the right and left CBCT images) generated by the stereoscopic image generating device (image processor 11), respectively. Moreover, the images (right and left images subjected to the superimposition) after the superimposition with the superimposing device (image processor 11) are displayed on the display device 14 (the monitor 14D in Embodiment 1) in real time. The three-dimensional coordinate detecting device (controller 15 in Embodiment 1) calculates and detects the three-dimensional coordinate of the object (the tip 23 of the bronchial endoscope 21 in Embodiment 1) from the position of the object displayed on the monitor in real time in accordance with the stereogram (the right and left fluoroscopy images) generated by the stereogram generating device (image processor 11).

As mentioned above, both the three-dimensional image (stereoscopic image) and the fluoroscopy image (stereogram) are same in terms of an X-ray image. Accordingly, these images are superimposed on each other to be displayed in real time, whereby a position and a direction of the object under fluoroscopy can be identified. Moreover, the three-dimensional coordinate is detected from the stereogram in real time. This facilitates identification of the position and the direction of the object under fluoroscopy, causing accurate navigation.

In Embodiment 1 as well as Embodiment 3, the stereogram generating device (image processor 11) generates the stereogram formed by the two fluoroscopy images (right and left fluoroscopy images). The fluoroscopic images are obtained through fluoroscopy in real rime with parallax in the projection directions (the A and B directions in each embodiment). That is, fluoroscopy for stereogram obtains the two fluoroscopy images (right and left fluoroscopy images) with parallax in real time for every fluoroscopy, whereby the stereogram is generated.

In the C-arm fluoroscopy apparatus according to Embodiment 1 as well as Embodiments 2 and 3, the three-dimensional coordinate detecting device (controller 15) detects the position of the tip of the inserting member (the bronchial endoscope 21 in the embodiments) as the three-dimensional coordinate, the inserting member being inserted into the subject M to be subjected to diagnosis or treatment. When fluoroscopy is conducted while the inserting member, typified by the bronchial endoscope 21, a catheter, a wire, and a source-inserting applicator, is inserted into the subject M, the position and direction of the inserting member (bronchial endoscope 21) can be identified readily under fluoroscopy without electricity and magnetism currently used. Here, in Embodiment 1 as well as Embodiments 2 and 3, the inserting member corresponds to the bronchial endoscope 21.

### EMBODIMENT 2

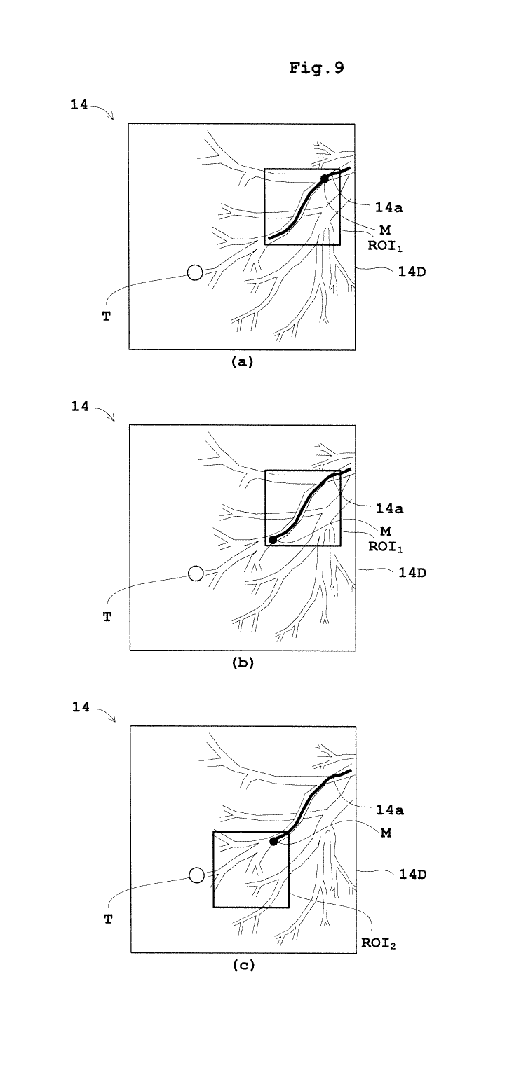

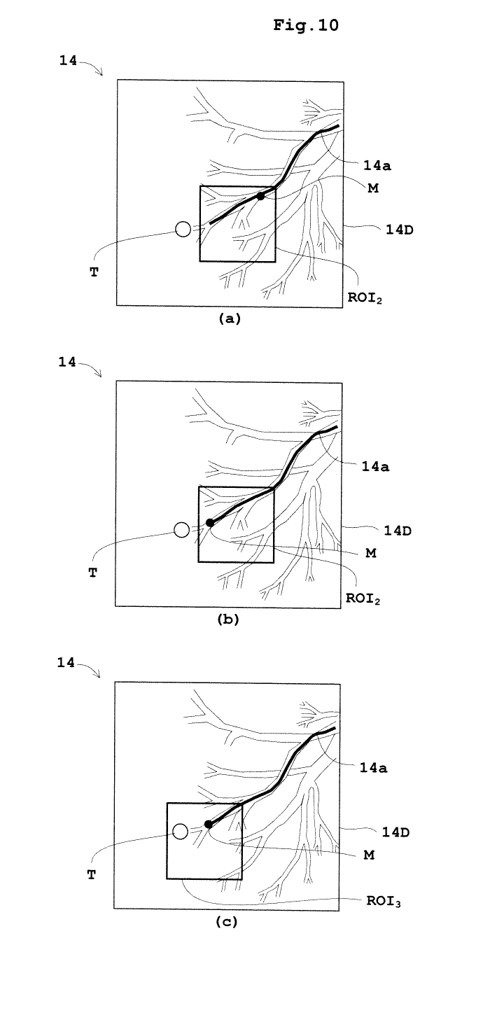

The following describes Embodiment 2 with reference to drawings. FIG. 7 is a flow chart illustrating a series of navigations according to Embodiment 2. FIGS. 8 to 11 are schematic views each illustrating one aspect of a display unit according to Embodiment 2. Parts in common with Embodiment 1 above are denoted by the same numerals, and descriptions thereof are to be omitted. In addition, as illustrated in FIG. 1, a C-arm fluoroscopy apparatus according to Embodiment 2 has the same construction as the C-arm fluoroscopy apparatus according to Embodiment 1.

In the Embodiment 1 mentioned above, the superimposition is performed to the entire image. In contrast to this, in Embodiment 2, a local region of interest (ROI: Region Of Interest) is selected for superimposition from the entire image in the three-dimensional image (the CBCT volume data) obtained in advance based on X-rays through cone-beam CT imaging (CBCT imaging) prior to endoscopy (fluoroscopy). Moreover, Embodiment 1 restricts the fluoroscopy image to the stereogram. In contrast to this, Embodiment 2 has no necessity of restricting the fluoroscopy image to the stereogram. That is, X-rays may be emitted from one focus and be detected with the X-ray detector 3, as illustrated in FIG. 2(a), for obtaining the fluoroscopy image through fluoroscopy. Similar to Embodiment 1, Embodiment 3 mentioned later restricts the fluoroscopy image to the stereogram.

As described in Operation and Effect in “Summary” of the latter embodiment, when fluoroscopy is conducted while the inserting member typified by the bronchial endoscope is inserted, the entire image is not so important, but merely the region of interest is needed. Consequently, the fluoroscopy image is superimposed on the three-dimensional image (the CBCT volume data) in the region of interest (ROI), causing sufficient identification of the position and direction of the tip of the bronchial endoscope. This is the reason why Embodiment 2 does not always need to restrict the fluoroscopy image to the stereogram. However, for more accurate identification of the position and direction of the bronchial endoscope in the fluoroscopy image, it is more preferable to apply the stereogram as the fluoroscopy image as in Embodiment 3 mentioned later.

Moreover, the C-arm fluoroscopy apparatus in Embodiment 2 has a function of setting and resetting the local region of interest (ROI). The controller 15 (see FIG. 1) may have the function of setting and resetting the region of interest. That is, the entire FIG. 14a (see FIG. 5) of the bronchial endoscope 21 (FIG. 6) has pixel values extremely different from therearound. Consequently, the controller 15 may set and reset while automatically calculating the region of interest (ROI) that tracks insertion of the bronchial endoscope 21. Of course, the input unit 13 (see FIG. 1) may have the function of setting and resetting the region of interest. That is, an operator recognizes the FIG. 14c (see FIG. 5) of the tip 23 (see FIG. 6) of the bronchial endoscope 21, and manually inputs a position of the tip 23 through the input unit 13 (see FIG. 1) by putting a pointer on a position of the monitor corresponding to the FIG. 14c. Consequently, the region of interest (ROI) is set and reset manually so as to contain the position. Such may be adopted. Moreover, combination of manual control and automatic control may be adopted.

Moreover, the following may be adopted. That is, the controller 15 has the function of setting the region of interest, and the input unit 13 has the function of resetting the region of interest, whereby the region of interest (ROI) is set automatically whereas the region of interest (ROI) is reset manually that tracks insertion of the bronchial endoscope 21. On the other hand, the following may also be adopted. That is, the input unit 13 has the function of setting the region of interest, and the controller 15 has the function of resetting the region of interest. Accordingly, the region of interest (ROI) is set manually whereas the region of interest (ROI) is reset automatically that tracks insertion of the bronchial endoscope 21. In the case of setting the region of interest (ROI) automatically, the controller 15 corresponds to the region of interest setting device. In the case of setting the region of interest (ROI) manually, the input unit 13 corresponds to the region of interest setting device. In combination of manual and automatic set of the region of interest (ROI), the input unit 13 and the controller 15 correspond to the region of interest setting device. Moreover, in the case of resetting the region of interest (ROI) automatically, the controller 15 corresponds to the region of interest resetting device. In the case of resetting the region of interest (ROI) manually, the input unit 13 corresponds to the region of interest resetting device. In combination of the manual and automatic reset of the region of interest (ROI), the input unit 13 and the controller 15 correspond to the region of interest resetting device.

In addition, the C-arm fluoroscopy apparatus in Embodiment 2 further has a function of shifting the stereoscopic image in the projection directions in the fluoroscopy image in synchronization with shifting of the fluoroscopy image in the region of interest (ROI), the stereoscopic image being based on the three-dimensional image (the CBCT volume data). Typically, a tissue and a structure inside the body contract and expand due to a body motion (e.g., a body motion by respiration) of the subject M (see FIGS. 1 and 2). In contrast to this, the contraction and expansion is not regarded within the local region of interest (ROI), and thus the image is shifted having a constant size of the tissue or the structure. Accordingly, the image processor 11 (see FIG. 1) calculates a shift amount of the stereoscopic image in synchronization with shifting of the fluoroscopy image in the region of interest (ROI), thereby shifting the stereoscopic image.

The image processor 11 in Embodiment 2 superimposes the fluoroscopy image on the shifted stereoscopic image in the region of interest (ROI). The display unit 14 (see FIG. 1) has the same construction as that in Embodiment 1, and description thereof is to be omitted. The image processor 11 in Embodiment 2 corresponds to the image shifting device in the present invention. The image processor 11 also corresponds to the superimposing device in the present invention. The display unit 14 corresponds to the display device in the present invention. The controller 15 corresponds to the three-dimensional coordinate detecting device in the present invention.

A series of navigations is performed along with the flow chart in FIG. 7. In FIGS. 8 to II, a lesion (e.g., a tumor) is denoted by a numeral T (see a mark “∘”).

(Step S1) Start Inserting Bronchial Endoscope

Firstly, the bronchial endoscope 21 (see FIG. 6) is inserted into a body (an oral cavity and a bronchus) of the subject M (see FIGS. 1 and 2) to start inserting the bronchial endoscope 21. The bronchial endoscope 21 travels into the body while monitoring in real time a main bronchus by capturing an image viewed from the lumen of the bronchus with an imaging element of the bronchial endoscope 21. In parallel with this, a fluoroscopy image of the bronchus is displayed in real time on the monitor 14D of the display unit 14 as illustrated in FIG. 8(a). At this time, an entire FIG. 14a of the bronchial endoscope 21 traveling inside the main bronchus is also displayed on the monitor 14D in real time. This continues until the bronchial endoscope 21 cannot travel any more. The Step S1 is performed prior to superimposition. Consequently, the image and the figure above may be displayed in real time on the monitor 14C in FIG. 5. Here, as illustrated in FIG. 8(a), the thin (e.g., peripheral) bronchus is invisible under typical fluoroscopy.

(Step S2) CBCT Imaging

When the bronchial endoscope 21 cannot travel any more, cone-beam CT imaging (CBCT imaging) is performed to obtain a plurality of projected images. Thereafter, the projected images are reconstructed three-dimensionally to generate a three-dimensional image (CBCT volume data).

As long as a position to which the bronchial endoscope 21 is inserted can be recognized, the three-dimensional image captured in advance without inserting the bronchial endoscope 21 or the three-dimensional image captured with the bronchial endoscope 21 removed therefrom is usable. This causes reduced artifacts or interference to X-rays by the bronchial endoscope 21.

(Step S3) Endoscopy

After the cone-beam CT imaging (CBCT imaging) in Step S2, the imaging system 4 constituted by the X-ray tube 2 and the X-ray detector 3 (both see FIGS. 1 and 2) is moved so as to conduct fluoroscopy to the thin bronchus, whereby a fluoroscopy image of the thin bronchus is displayed on the monitor 14D of the display unit 14 in real time as illustrated in FIG. 8(b). At this time, the entire image 14a of the bronchial endoscope 21 that cannot travel any more is also displayed on the monitor 14D in real time. The endoscopy (fluoroscopy) is conducted in this manner.

(Step S4) Set and Reset ROI

The controller 15 (see FIG. 1) then automatically sets a local region of interest (ROI). Alternatively, the operator uses the input unit 13 (see FIG. 1) to manually put a pointer on a portion of the monitor corresponding to the FIG. 14c (see FIG. 5) of the tip 23 (see FIG. 6) of the bronchial endoscope 21, thereby setting the region of interest (ROI) manually. The size of the region of interest (ROI) is not particularly limited. The size containing a forward bifurcation of the bronchus is preferable. In FIG. 8(c), a region of interest (ROI) set primarily is denoted by a numeral ROI₁, and a landmark on the clamp extending from the tip 23 is denoted by a numeral M (see the mark “”).

In the set region of interest ROI₁, the image processor 11 (see FIG. 1) calculates a shift amount of the stereoscopic image, obtained through the cone-beam CT imaging (CBCT imaging) in Step S2 based on the three-dimensional image, so as to correspond to the shifting of the fluoroscopy images, whereby the stereoscopic image is shifted. Moreover, in the region of interest ROI₁, the fluoroscopy image is superimposed on the shifted stereoscopic image, and the image subjected to the superimposition (the image after the superimposition) is displayed on the monitor 14D in real time (hereinafter, abbreviated to “shift display”).

The shift display is repeated for several-time respiratory. The display is made at a frame rate in synchronization with a period for respiratory, whereby the period is displayed while being locked (fixed). Accordingly, the image after the superimposition is fixedly displayed on the monitor 14D in the same position. At this time, the landmark M is marked on the clamp. Here, the marking may be performed with the controller 15 automatically, or with the input unit 13 manually.

The controller 15 calculates and detects the three-dimensional coordinate of the object (the tip 23 of the bronchial endoscope 21) from the position of the object displayed on the monitor in real time in accordance with the three-dimensional image (stereoscopic image) and the fluoroscopy image in the region of interest ROI₁.

Here, the position and the direction of the bronchial endoscope 21 can be identified with the shift display. Consequently, the bronchial endoscope 21 can travel again into the thin bronchus. After travelling while being displayed in real time, the bronchial endoscope 21 stops travelling at a forward bifurcation of the bronchus, as illustrated in FIG. 9(a). The landmark M in FIG. 9(a) stays at a position corresponding to that in FIG. 8(c). Alternatively, a forward bifurcation of the bronchus is marked with a landmark M, where the bronchial endoscope 21 may stop travelling. Then, as illustrated in FIG. 9(b), the clamp extending from the tip 23 of the bronchial endoscope 21 is again marked with the landmark M.

At this time, the three-dimensional coordinate (of the clamp extending from the tip 23 of the bronchial endoscope 21) displayed in real time is likely to go beyond the region of interest ROI₁. Note that “go beyond the region of interest” in the specification includes not only the case of going beyond the region of interest actually, but also the case of almost going beyond the region of interest. When the three-dimensional coordinate of the clamp is likely to go beyond the region of interest ROI₁, the region of interest (ROI) is reset so as to contain the three-dimensional coordinate entirely.

Similar to setting of the region of interest ROI₁, the controller 15 resets the region of interest (ROI) automatically. Alternatively, the operator uses the input unit 13 to put a pointer manually on a position on the screen corresponding to the FIG. 14c of the tip 23 of the bronchial endoscope 21, thereby resetting the region of interest (ROI) manually. In FIG. 9(c), a new reset region of interest (ROI) is denoted by a numeral ROI₂.

Similar to the setting of the region of interest ROI₁, shifting the images, superimposition, monitoring to the display unit 14, and detecting the three-dimensional coordinate are repeatedly conducted to perform shift display in the reset region of interest ROI₂.

The bronchial endoscope 21 again travels with the shift display. After travelling while being displayed in real time, the bronchial endoscope 21 stops travelling at a forward bifurcation of the bronchus, as illustrated in FIG. 10(a). Then, as illustrated in FIG. 10(b), the clamp extending from the tip 23 of the bronchial endoscope 21 is again marked with the landmark M.

Similarly, the three-dimensional coordinate of the clamp displayed in real time is likely to go beyond the region of interest ROI₂. When the three-dimensional coordinate of the clamp is likely to go beyond the region of interest ROI₂, the region of interest (ROI) is reset so as to contain the three-dimensional coordinate entirely.

Similar to setting of the region of interest ROI₁ and resetting of the region of interest ROI₂, the controller 15 resets the region of interest (ROI) automatically. Alternatively, the operator uses the input unit 13 to put a pointer manually on a position on the screen corresponding to the FIG. 14c of the tip 23 of the bronchial endoscope 21, thereby resetting the region of interest (ROI) manually. In FIG. 10(c), a new reset region of interest (ROI) is denoted by a numeral ROI₃.

Similar to the setting of the region of interest ROI₁ and resetting of the region of interest ROI₂, shifting the images, superimposition, monitoring to the display unit 14, and detecting the three-dimensional coordinate are repeatedly conducted to perform shift display in the reset region of interest ROI₃.

The bronchial endoscope 21 again travels with the shift display. After travelling while being displayed in real time, the bronchial endoscope 21 stops travelling at a forward bifurcation of the bronchus, as illustrated in FIG. 11. As noted above, shifting the images, superimposition, monitoring to the display unit 14, and detecting the three-dimensional coordinate are repeatedly conducted in the reset region of interest (ROI), whereby the shift display is performed repeatedly.

(Step S5) Reach Tumor?

Then it is determined whether or not the clamp extending from the tip 23 of the bronchial endoscope 21 reaches a tumor T. In actual, the bronchial endoscope 21 does not possibly reach a lesion typified by the tumor T, or the lesion is invisible through the bronchial endoscope 21. Accordingly, the clamp inserted through the bronchial endoscope 21 may stop in front of the lesion, or may pass through the lesion without stopping when there is patency of the bronchus in the lesion. In such a case, it is preferable to confirm the tip of the clamp within the lesion three-dimensionally with the fluoroscopy images obtained through the X-ray fluoroscopy or the CT images (e.g., the right and left CBCT images) obtained through the CT imaging. The following is a description under an assumption that the clamp reaches the tumor T.

This confirmation may be made with the controller 15 automatically, or with the input unit 13 manually. When the clamp does not reach the tumor T, the process returns to the Step S3, and then the Step S4 of resetting the ROI including the shift display and the Step S5 of determining reach to the tumor are repeatedly performed. When the clamp reaches the tumor T as illustrated in FIG. 11, a series of navigations is completed. Thereafter, the clamp collects a tissue (the tumor T in the embodiment) for biopsy.

With the C-arm fluoroscopy apparatus according to Embodiment 2, the region of interest setting device (the input unit 13 or the controller 15 in Embodiment 2) sets the local region of interest (the ROI₁ in FIGS. 8 and 9). The image shifting device (the image processor 11 in Embodiment 2) shifts the stereoscopic image in synchronization with shifting of the fluoroscopy image in the region of interest ROI₁ set with the region of interest setting device (the input unit 13 or the controller 15), the stereoscopic image being obtained based on the three-dimensional images obtained in advance in accordance with X-rays in the projection directions of the fluoroscopy images (obtained based on the X-rays). Typically, the tissue and structure inside the body contract and expand due to the body motion (e.g., the body motion by respiration) of the subject M. In contrast to this, the contraction and expansion is not regarded within the local region of interest (ROI), and thus the image is shifted having a constant size of the tissue or structure. Moreover, when fluoroscopy is conducted while the inserting member (the bronchial endoscope 21 in each embodiment) is inserted, the entire image is not so important, but merely the region of interest (ROI) is needed.

Then, the stereoscopic image can be shifted in the region of interest (ROI) in synchronization with shift of the fluoroscopy image. Moreover, for the body motion due to the respiration, the three-dimensional image (the CBCT volume data) in synchronization with a respiration sensor or the three-dimensional image (the CBCT volume data) in synchronization with each of a plurality of phases is obtained in advance to deal with superimposition by the body motion. Such a mode is conceivable. However, the mode needs the respiration sensor, or increases radiographic frequency for obtaining an image for each of the plurality of phases, causing an increased inspection e, an exposure radiation dose, or a processing time. Consequently, the mode is not practical. In addition, large movement of the subject M causes all the images to be taken again, leading to much waste. Embodiment 2 differs from the mode in which a deviation amount of the stereoscopic image is calculated in accordance with a deviation amount of the fluoroscopy image upon changing the projection direction for superimposing both the images on each other. In Embodiment 2, the stereoscopic image is simply shifted under an assumption that the image has an uniform size of the tissue or structure within the region of interest (ROI). Accordingly, the currently-used respiration sensor is not needed. In addition, a radiographic frequency, an exposure radiation dose, an inspection time, and a processing time can be reduced without obtaining the three-dimensional image in advance in synchronization with each of a plurality of phases.

Then, the superimposing device (the image processor 11 in Embodiment 2) superimposes the fluoroscopy image on the stereoscopic image shifted by the image shifting device (image processor 11) in the region of interest (ROI). The image after superimposed with the superimposing device (image processor 11) is displayed on the display device (the monitor 14D of the display unit 14 in Embodiment 2) in real time. The three-dimensional coordinate detecting device (the controller 15 in Embodiment 2) calculates and detects the three-dimensional coordinate of the object (the tip 23 of the bronchial endoscope 21 in Embodiment 2) from the position of the object displayed in real time on the screen in accordance with the three-dimensional image and fluoroscopy image in the region of interest (the ROI₁ in FIGS. 8 and 9).

As mentioned above, both the three-dimensional image (stereoscopic image) and the fluoroscopy image are same in terms of an X-ray image. Accordingly, these images are superimposed on each other to be displayed in real time, whereby a position and a direction of the object under fluoroscopy can be identified. Moreover, the three-dimensional coordinate is detected from the three-dimensional image and the stereogram in real time. This facilitates identification of the position and the direction of the object under fluoroscopy, causing accurate navigation.

Embodiment 2 includes a region of interest resetting device (the input unit 13 or the controller 15 in Embodiment 2) configured to reset the region of interest so as to be located within the three-dimensional coordinate when the three-dimensional coordinate displayed in real time goes beyond the region of interest (ROI₁ to ROI₃ in FIGS. 8 to 11). The image shifting device (the image processor 11), the superimposing device (the image processor 11), the display device (the monitor 14D of the display unit 14), and the three-dimensional coordinate detecting device (the controller 15) each repeatedly perform its processing to the region of interest reset by the region of interest resetting device (the input unit 13 or the controller 15 in Embodiment 2). Such is preferable.

The image shifting device (the image processor 11), the superimposing device (the image processor 11), the display device (the monitor 14D of the display unit 14), and the three-dimensional coordinate detecting device (the controller 15) each repeatedly perform its processing to the region of interest ROI₂, ROI₃ reset by the region of interest resetting device (the input unit 13 or the controller 15). This achieves navigation while tracking the variable three-dimensional coordinate for example when fluoroscopy is conducted with the inserting member (bronchial endoscope 21) being inserted. Moreover, the region of interest (ROI) tracks the coordinate during the navigation while being reset repeatedly. This achieves accurate navigation while tracking the coordinate.

### EMBODIMENT 3

The following describes Embodiment 3 with reference to drawings. Parts in common with Embodiments 1 and 2 above are denoted by the same numerals, and description thereof is to be omitted. Moreover, as illustrated in FIG. 1, the C-arm fluoroscopy apparatus in Embodiment 3 has the same construction as that according to Embodiments 1 and 2.

Embodiment 3 is a combination of Embodiments 1 and 2 above.

That is, Embodiment 3 has the construction including the stereogram generating device (the image processor 11 in Embodiment 1) and the stereoscopic image generating device (the image processor 11 in Embodiment 1) in Embodiment 2. The fluoroscopy image is restricted to the stereogram in the image shifting device (image processor 11) in Embodiment 2. Accordingly, the image shifting device (image processor 11) shifts the stereoscopic image generated by the stereoscopic image generating device (image processor 11) in synchronization with the shifting of the stereogram in the region of interest (ROI) in Embodiment 3.

Moreover, the fluoroscopy image is restricted to the stereogram in the superimposing device (image processor 11 in Embodiment 2) in Embodiment 2. Accordingly, the superimposing device (image processor 11) superimposes the stereogram on the stereoscopic image shifted by the image shifting device for the projection directions in the region of interest (ROI) in Embodiment 3. In other words, in the superimposing device in Embodiment 1 (the image processor 11 also in Embodiment 1), a processed portion is restricted to the region of interest (ROI), whereby the stereogram is superimposed on the stereoscopic image shifted by the image shifting device (the image processor 11) for every projection direction in the region of interest (ROI) in Embodiment 3.

Similar to the display device (the monitor 14D of the display unit 14) in Embodiment 1, the display device (the monitor 14D of the display unit 14 in Embodiment 2) in Embodiments 2 and 3 displays the images superimposed with the superimposing device (image processor 11) in real time. In the three-dimensional coordinate detecting device in Embodiment 2 (the controller 15 in Embodiment 2), the fluoroscopy image is restricted to the stereogram. Accordingly, in Embodiment 3, the three-dimensional coordinate detecting device (controller 15) calculates and detects the three-dimensional coordinate based on the three-dimensional image and the stereogram in the region of interest (ROI). In other words, in the three-dimensional coordinate detecting device in Embodiment 1 (the controller 15 also in Embodiment 1), the processed portion is restricted to the region of interest (ROI) and the three-dimensional image is added to base data besides the stereogram. Consequently, in Embodiment 3, the three-dimensional coordinate is calculated and detected based on the three-dimensional image and the stereogram in the region of interest (ROI). Since the other operations and effects are produced from the combination of Embodiments 1 and 2, the description thereof is to be omitted.

Similar to Embodiment 1, fluoroscopy for stereogram is conducted in Embodiment 3, whereby the two fluoroscopy images (right and left fluoroscopy images) are obtained with parallax in real time for every fluoroscopy to generate the stereogram.

The present invention is not limited to the foregoing embodiments, but may be modified as under.

(1) Each embodiment mentioned above is applied to the C-arm fluoroscopy apparatus as illustrated in FIG. 1. Alternatively, each embodiment is applicable to a fluoroscopy apparatus having an imaging system fixed on the ceiling or a side wall, or to a surgical X-ray apparatus. Moreover, an apparatus is applicable having an arrangement of the X-ray tube and the X-ray detector, forming the imaging system, replaced by each other.

(2) In each embodiment mentioned above, the bronchial endoscope is inserted into the bronchus of the subject for diagnosis on the bronchus. Alternatively, with a medical X-ray apparatus for diagnosis or treatment to the subject, diagnosis or treatment may be conducted by inserting the catheter or wire into the blood vessel to a target site as in blood vessel contrast radiography. Moreover, the source inserting applicator may be inserted to a treatment site for radiation treatment plan with a source or a dummy source. For instance, when a particle (also referred to as a “seed”) is embedded inside the body, a treatment plan is conceivable for positioning a seed to be inserted in accordance with the embedded seed.

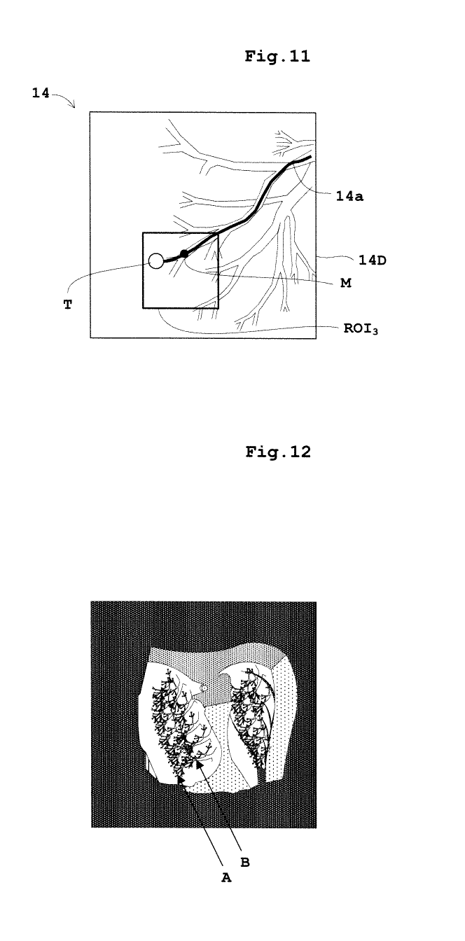

(3) Each embodiment mentioned above adopts a method of providing parallax with the crossing method as in FIG. 4. Alternatively, the parallax may be provided with the parallel method as in FIG. 12.

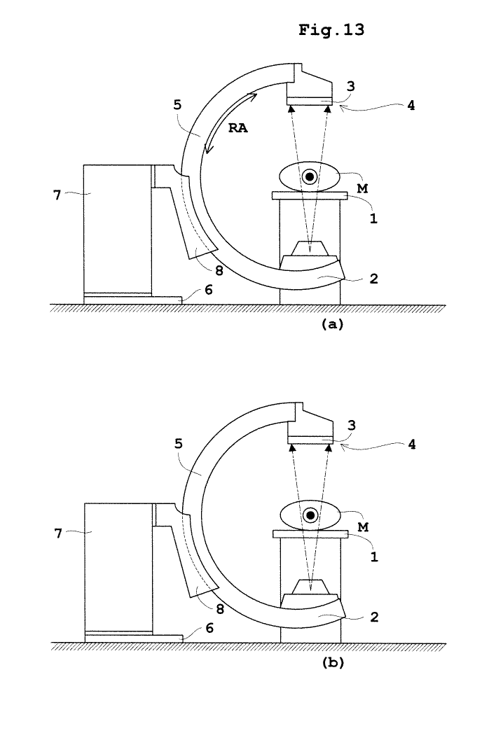

(4) In each embodiment mentioned above, the stereo X-ray tube as in FIG. 2 that switches the focuses with pulses is adopted as the X-ray tube 2. Alternatively, the typical X-ray tube 2 in FIG. 13 that includes one focus may be adopted. For obtaining the three-dimensional image, the imaging system 4 is moved in each direction (e.g., is rotated by approximately 200 degrees in the RA arrow direction) as illustrated in FIG. 13(a). For obtaining the fluoroscopy image, the fluoroscopy image may be obtained in real time with no parallax, as illustrated in FIG. 13(b). The construction in FIG. 13 is useful when no restriction is performed to the stereogram as in Embodiment 2.

(5) In Embodiments 1 and 3, when fluoroscopy for the stereogram is conducted, the two fluoroscopy images are obtained with parallax in real time to generate the stereogram. However, the fluoroscopy for stereogram is not limitative. For instance, a stereogram may be generated from one original fluoroscopy image obtained through fluoroscopy in real time. Here, the stereogram is formed by the original fluoroscopy image and a fluoroscopy image based on the three-dimensional image obtained with the construction of FIG. 2(a) or 13(a) with parallax in a projection direction of the original fluoroscopy image. That is, typical fluoroscopy (not the fluoroscopy for stereogram) is conducted, whereby one original fluoroscopy image is obtained in real time for every fluoroscopy. Then, the stereogram is generated from the original fluoroscopy image, the stereogram being formed by the original fluoroscopy image and the fluoroscopy image with parallax in the projection direction of the original fluoroscopy image. In this case, the stereogram can be generated by the typical X-ray tube 2, as in FIG. 13, having one focus.

(6) In each embodiment mentioned above, the same apparatus as in FIG. 2 is used for obtaining the fluoroscopy image and for obtaining the three-dimensional image. Alternatively, the medical X-ray apparatus is used only for fluoroscopy, and another apparatus (an external apparatus) typified by the X-ray CT apparatus is used for obtaining the three-dimensional image. Such may be adopted. However, it is preferable to use the same apparatus in terms of successive radiography and fluoroscopy or accurate navigation.

(7) In Embodiments 2 and 3 mentioned above, the display position in the fluoroscopy image and the stereogram is fixed, and the stereoscopic image in the region of interest (ROI) is shifted in synchronization with shifting of the images. Then the fluoroscopy image or the stereogram is superimposed on the shifted stereoscopic image. Alternatively, an order reverse to this may be adopted. That is, the display position in the stereoscopic image is fixed, and the fluoroscopy image or the stereogram is shifted in the region of interest (ROI) in synchronization with the display position in the fixed stereoscopic image. Then the fluoroscopy image or the stereogram is superimposed on the shifted stereoscopic image. Such may be adopted. In this case, the fluoroscopy image or the stereogram is shifted in correspondence to the fixed position in the stereoscopic image even when the fluoroscopy image or the stereogram is shifted. Accordingly, if the fluoroscopy image or the stereogram is shifted, the fluoroscopy image or the stereogram is always located on the fixed position in the stereoscopic image, whereby the fluoroscopy image or the stereogram seems to be stable. In addition, in Embodiment 2, display of the period is locked (fixed). With the modification (7), an effect is also produced that the image after the superimposition can be displayed at smaller frame rates.

(8) In Embodiments 2 and 3 includes the region of interest resetting device configured to reset the region of interest so as the region of interest to contain the three-dimensional coordinate entirely when the three-dimensional coordinate displayed in real time goes beyond the region of interest (ROI₁ to ROI₃). Alternatively, the region of interest resetting device is not always needed when the three-dimensional coordinate is not tracked.

### REFERENCE SIGN LIST

11 . . . image processor
13 . . . input unit
14 . . . display unit
14D . . . monitor
15 . . . controller
21 . . . bronchial endoscope
ROI . . . region of interest
M . . . subject

1. A medical X-ray apparatus configured to perform diagnosis or treatment by displaying a fluoroscopy image in real time in accordance with detected X-rays, the medical X-ray apparatus comprising:
a stereogram generating device configured to generate a stereogram formed by two fluoroscopy images with parallax in projection directions;
a stereoscopic image generating device configured to generate a stereoscopic image in the projection directions in the stereogram generated by the stereogram generating device based on a three-dimensional image obtained in advance in accordance with X-rays;
a superimposing device configured to perform superimposition of the stereogram in the projection directions on the stereoscopic image generated by the stereoscopic image generating device;
a display device configured to display an image subjected to the superimposition in real time by the superimposing device; and
a three-dimensional coordinate detecting device configured to calculate and detect a three-dimensional coordinate of an object from a position of the object displayed on a screen of the display device in real time in accordance with the stereogram generated by the stereogram generating device.

2. A medical X-ray apparatus configured to perform diagnosis or treatment by displaying a fluoroscopy image in real time in accordance with on detected X-rays, the medical X-ray apparatus comprising:
a region of interest setting device configured to set a local region of interest;
an image shifting device configured to (1) shift a stereoscopic image in a projection direction of the fluoroscopy image in the region of interest set by the region of interest setting device in synchronization with shifting of the fluoroscopy image, the stereoscopic image being based on a three-dimensional image obtained in advance in accordance with the X-rays, or configured to (2) shift the fluoroscopy image in the region of interest set by the region of interest setting device in synchronization with a position of a stereoscopic image in a projection direction of the fluoroscopy image, the stereoscopic image being fixed and being based on a three-dimensional image obtained in advance in accordance with the X-rays;
a superimposing device configured to perform superimposition of (1) the fluoroscopy image on the stereoscopic image shifted by the image shifting device, or (2) the stereoscopic image on the fluoroscopy image shifted by the image shifting device in the region of interest;
a display device configured to display an image subjected to the superimposition in real time by the superimposing device; and
a three-dimensional coordinate detecting device configured to calculate and detect a three-dimensional coordinate of an object from a position of the object displayed on a screen of the display device in real time in accordance with the three-dimensional image and the fluoroscopy image in the region of interest.

3. The medical X-ray apparatus according to claim 2, further comprising:
a region of interest re-setting device configured to reset a region of interest so as to contain the three-dimensional coordinate displayed in real time when the three-dimensional coordinate goes beyond the region of interest, wherein
the image shifting device, the superimposing device, the display device, and the three-dimensional coordinate detecting device each repeatedly perform its processing to the region of interest reset by the region of interest resetting device.

4. The medical X-ray apparatus according to claim 2, further comprising:
a stereogram generating device configured to generate a stereogram formed by two fluoroscopy images having parallax in projection directions; and
a stereoscopic image generating device configured to generate a stereoscopic image based on a three-dimensional image in the projection directions of the stereogram generated by the stereogram generating device, wherein
the image shifting device (1) shifts the stereoscopic image generated by the stereoscopic image generating device in the region of interest in synchronization with shifting of the stereogram, or (2) shifts the stereogram in synchronization with a position of the stereoscopic image in the region of interest, the stereoscopic image being fixed by the stereoscopic image generating device,
the superimposing device performs superimposition of (1) the stereogram on the stereoscopic image shifted by the image shifting device for every projection direction, or (2) the stereoscopic image on the stereogram shifted by the image shifting device for every projection direction in the region of interest,
the display device displays an image subjected to the superimposition by the superimposing device in real time, and
the three-dimensional coordinate detecting device calculates and detects the three-dimensional coordinate based on the three-dimensional image and the stereogram in the region of interest.

5. The medical X-ray apparatus according to claim 1, wherein
the stereogram generating device generates the stereogram formed by two fluoroscopy images obtained through fluoroscopy in real rime with parallax in the projection directions.

6. The medical X-ray apparatus according to claim 1, wherein
the stereogram generating device generates the stereogram from one original fluoroscopy image obtained through fluoroscopy in real time, the stereogram being formed by the original fluoroscopy image and a fluoroscopy image based on the three-dimensional image with parallax in a projection direction of the original fluoroscopy image.

7. The medical X-ray apparatus according to claim 1, wherein
the three-dimensional coordinate detecting device detects a position of a tip of an inserting member as the three-dimensional coordinate, the inserting member being inserted into a subject to which diagnosis or treatment is performed.

8. The medical X-ray apparatus according to claim 7, wherein
the inserting member is an endoscope, a source-inserting applicator, a dummy source, or a catheter wire.

9. The medical X-ray apparatus according to claim 4, wherein
the stereogram generating device generates the stereogram formed by two fluoroscopy images obtained through fluoroscopy in real rime with parallax in the projection directions.

10. The medical X-ray apparatus according to claim 4, wherein
the stereogram generating device generates the stereogram from one original fluoroscopy image obtained through fluoroscopy in real time, the stereogram being formed by the original fluoroscopy image and a fluoroscopy image based on the three-dimensional image with parallax in a projection direction of the original fluoroscopy image.

11. The medical X-ray apparatus according to claim 2, wherein
the three-dimensional coordinate detecting device detects a position of a tip of an inserting member as the three-dimensional coordinate, the inserting member being inserted into a subject to which diagnosis or treatment is performed.

12. The medical X-ray apparatus according to claim 11, wherein
the inserting member is an endoscope, a source-inserting applicator, a dummy source, or a catheter wire.
